(19) United States
(12) Patent Application Publication
(10) Pub. No.: US 2025/0032756 A1
(43) Pub. Date: Jan. 30, 2025

(54) LOCKING MECHANISM FOR LOOPED CATHETER

(71) Applicant: Boston Scientific Scimed, Inc., Maple Grove, MN (US)

(72) Inventors: Ajay Gupta, Minneapolis, MN (US); Randy Raymond, Maple Grove, MN (US)

(73) Assignee: Boston Scientific Scimed, Inc., Maple Grove, MN (US)

(21) Appl. No.: 18/779,805

(22) Filed: Jul. 22, 2024

Related U.S. Application Data
(60) Provisional application No. 63/528,777, filed on Jul. 25, 2023.

(51) Int. Cl. A61M 25/04; A61M 25/01

(52) U.S. Cl. CPC A61M 25/04; A61M 2025/0163

(57) ABSTRACT

Locking catheters including a fluid-tight hub and mechanism to secure a flexible member. An illustrative locking catheter may comprise an elongate shaft defining a lumen, a first connection member coupled to the elongate shaft, a second connection member configured to be releasably coupled to the first connection member, and a flexible member extending through at least a portion of a lumen of the elongate shaft and a portion of a lumen of the first connection member. The proximal end of the flexible member may extend through the aperture of the first connection member such that the proximal end of the flexible member is exterior to the first connection member. When the second connection member is coupled with the first connection member, the flexible member may be secured between an inner surface of the first connection member and an outer surface of the inner tubular member of the second connection member.

### CROSS-REFERENCE TO RELATED APPLICATIONS

This application claims the benefit of U.S. Provisional Patent Application Ser. No. 63/528,777, filed Jul. 25, 2023, which is incorporated herein by reference.

### TECHNICAL FIELD

The disclosure is directed to catheters. More particularly, the disclosure is directed to a locking mechanism for a looped catheter.

### BACKGROUND

Typically, drainage catheters are tubular, flexible conduits percutaneously inserted into a fluid collection within the viscera. Common applications of drainage catheters include abscess, biliary, and nephrostomy drainage resulting from the body's temporary inability to naturally drain these fluid collections. To inhibit catheter movement, a pigtail loop or other retention structure may be formed at the catheter's distal end. The loop, once formed, engages surrounding tissue, such as the inner walls of a lumen or organ, thus preventing the catheter from displacing due to accidental tugging or pulling. Of the known systems for maintaining a loop or curve in a locking catheter, there is an ongoing need to provide alternative configurations of locking systems.

### SUMMARY

This disclosure provides design, material, manufacturing method, and use alternatives for medical devices.

In a first example, a locking catheter may comprise an elongate shaft extending from a proximal end to a distal end and defining a lumen extending from the proximal end to the distal end and a hub defining a lumen extending from a proximal end to a distal end thereof and defining a lumen including extending from the proximal end to the distal end. The hub may include a first connection member having a body portion extending from a proximal end to a distal end and defining an aperture extending through a sidewall of the body portion of the first connection member from an inner surface to an outer surface thereof. The hub may be coupled to the proximal end of the elongate shaft. The locking catheter may further comprise a second connection member extending from a proximal end to a distal end and having an outer coupling member and an inner tubular member, the inner tubular member defining a lumen extending from the proximal end to the distal end of the second connection member, the second connection member configured to be releasably coupled to the first connection member and a flexible member extending from a proximal end to a distal end, the distal end of the flexible member coupled to the elongate shaft, the flexible member extending through at least a portion of the lumen of the elongate shaft and a portion of the lumen of the hub, the proximal end of the flexible member extending through the aperture of the first connection member such that the proximal end of the flexible member is exterior to the first connection member. When the second connection member is coupled with the first connection member, the flexible member may be secured between an inner surface of the first connection member and an outer surface of the inner tubular member of the second connection member.

Alternatively or additionally to any of the examples above, in another example, when the second connection member is coupled with the first connection member, the second connection member and the first connection member may form a fluid-tight seal.

Alternatively or additionally to any of the examples above, in another example, the first connection member may comprise a female luer lock.

Alternatively or additionally to any of the examples above, in another example, the second connection member may comprise a male luer lock.

Alternatively or additionally to any of the examples above, in another example, the aperture of the first connection member may be in the range of about 0.09 inches (2.29 millimeters) to about 0.11 inches (2.79 millimeters) from the proximal end of the first connection member.

Alternatively or additionally to any of the examples above, in another example, the aperture of the first connection member may have a diameter in the range of about 0.010 inches (0.254 millimeters) to about 0.014 inches (0.356 millimeters).

Alternatively or additionally to any of the examples above, in another example, the locking catheter may further comprise a channel formed in the inner surface of the first connection member.

Alternatively or additionally to any of the examples above, in another example, the channel may extend distally from the aperture of the first connection member.

Alternatively or additionally to any of the examples above, in another example, a longitudinal axis of the aperture of the first connection member may extend generally orthogonal to a longitudinal axis of the elongate shaft.

Alternatively or additionally to any of the examples above, in another example, a longitudinal axis of the aperture of the first connection member may extend at a non-orthogonal to a longitudinal axis of the elongate shaft.

Alternatively or additionally to any of the examples above, in another example, actuation of the flexible member may be configured to move a distal end region of the elongate shaft into a curved configuration.

Alternatively or additionally to any of the examples above, in another example, the distal end of the flexible member may be coupled to the elongate shaft adjacent to the distal end of the elongate shaft.

Alternatively or additionally to any of the examples above, in another example, the distal end of the flexible member may be coupled to the elongate shaft adjacent to the proximal end of the elongate shaft.

Alternatively or additionally to any of the examples above, in another example, the locking catheter may further comprise a plurality of apertures extending through a sidewall of the elongate shaft, the plurality of apertures adjacent to the distal end of the elongate shaft.

Alternatively or additionally to any of the examples above, in another example, the outer coupling member of the second connection member may be configured to threadably engage the first connection member.

In another example, a locking catheter may comprise an elongate shaft extending from a proximal end to a distal end and defining a lumen extending from the proximal end to the distal end and a hub defining a lumen extending from a proximal end to a distal end thereof and defining a lumen including extending from the proximal end to the distal end. The hub may include a female luer lock having a body portion extending from a proximal end to a distal end and defining an aperture extending through a sidewall of the body portion of the female luer lock from an inner surface to an outer surface thereof. The hub may be coupled to the proximal end of the elongate shaft. The locking catheter may further comprise a male luer lock extending from a proximal end to a distal end and having an outer coupling member and an inner tubular member, the inner tubular member defining a lumen extending from the proximal end to the distal end of the male luer lock, the male luer lock configured to be releasably coupled to the female luer lock and a flexible member extending from a proximal end to a distal end, the distal end of the flexible member coupled to the elongate shaft, the flexible member extending through at least a portion of the lumen of the elongate shaft and a portion of the lumen of the hub, the proximal end of the flexible member extending through the aperture of the female luer lock such that the proximal end of the flexible member is exterior to the female luer lock. When the male luer lock is coupled with the female luer lock, the flexible member may be secured between an inner surface of the female luer lock and an outer surface of the inner tubular member of the male luer lock.

Alternatively or additionally to any of the examples above, in another example, the aperture of the female luer lock may be distal to the proximal end of the female luer lock.

Alternatively or additionally to any of the examples above, in another example, when the male luer lock is coupled with the female luer lock, the male luer lock and the female luer lock may form a fluid-tight seal.

Alternatively or additionally to any of the examples above, in another example, the 20) aperture of the female luer lock may be in the range of about 0.09 inches (2.29 millimeters) to about 0.11 inches (2.79 millimeters) from the proximal end of the female luer lock.

In another example, a locking catheter may comprise an elongate shaft extending from a proximal end to a distal end and defining a lumen extending from the proximal end to the distal end and a hub defining a lumen extending from a proximal end to a distal end thereof and defining a lumen including extending from the proximal end to the distal end. The hub may include a first connection member coupled to the proximal end of the elongate shaft, the first connection member having a body portion extending from a proximal end to a distal end and defining a slot extending through a thickness of a sidewall of the body portion, the slot extending distally from the proximal end of the first connection member. The locking catheter may further comprise a second connection member extending from a proximal end to a distal end and having an outer coupling member and an inner tubular member, the inner tubular member defining a lumen extending from the proximal end to the distal end of the second connection member, the second connection member configured to be releasably coupled to the first connection member and a flexible member extending from a proximal end to a distal end, the distal end of the flexible member coupled to the elongate shaft, the flexible member extending through at least a portion of the lumen of the elongate shaft and a portion of the lumen of the hub, the proximal end of the flexible member extending through the slot of the first connection member such that the proximal end of the flexible member is exterior to the first connection member. When the second connection member is coupled with the first connection member, the flexible member may be secured between an inner surface of the first connection member and an outer surface of the inner tubular member of the second connection member.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a side view of an illustrative catheter;

FIG. 2 is a perspective view of a proximal portion of the illustrative catheter of FIG. 1;

FIG. 3 is a cross-sectional view of the illustrative catheter of FIG. 1, taken at line 3-3 of FIG. 2;

FIG. 4 is an alternative cross-sectional view of the illustrative catheter of FIG. 1, taken at line 3-3 of FIG. 2;

FIG. 5 is a cross-sectional view of the proximal portion of the catheter, taken at line 5-5 of FIG. 1;

FIG. 6 is an enlarged cross-sectional view of region A in FIG. 5;

FIG. 7A is a schematic view of the looped catheter of FIG. 1 disposed within a patient in an unlocked position;

FIG. 7B is a schematic view of the looped catheter of FIG. 1 disposed within a patient in a locked position; and

FIG. 8 is a perspective view of a proximal portion of an alternative configuration of the illustrative catheter of FIG. 1.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. For purposes of this disclosure, “proximal” refers to the end closer to the device operator during use, and “distal” refers to the end further from the device operator during use.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Typically, drainage catheters are tubular, flexible conduits percutaneously inserted into a fluid collection within the viscera, such as, but not limited to the kidneys, bladder, abdominal cavity, stomach, biliary system, etc. Common applications of drainage catheters include, but are not limited to, abscess, biliary, and nephrostomy drainage resulting from the body's temporary inability to naturally drain these fluid collections. A drainage catheter may be introduced over a stiffening cannula using either a direct trocar stick or a Seldinger technique (e.g., over a guidewire). To inhibit catheter movement once placed, a pigtail loop, a curved end (such as a “J” shape), or other retention structure is often formed at the catheter's distal end. The retention structure, once formed, may engage surrounding tissue, such as the inner walls of a lumen or organ, thus preventing the catheter from displacing due to accidental tugging or pulling.

Some catheters may include a pre-formed pigtail loop at their distal end. Before placing this catheter in the body, a rigid wire, or other straightening member, is inserted within a lumen of the catheter to straighten out the pre-formed loop. Once the catheter is placed, the rigid wire is removed allowing the distal end of the catheter to resume the pre-formed pigtail loop. Alternatively, the pigtail loop may be formed after the catheter is inserted at the desired location. In such catheters, one end of a suture may be fixed to the catheter. The suture may extend along the catheter's length and exit from its proximal end. By drawing the suture proximally, the distal tip of the catheter may be forced to curl into a curved or pigtail formation. A proximal suture portion is then secured to hold the suture in place and retain the curved or looped shape at the distal end of the catheter. It is contemplated that in some cases, a locking hub may be used to secure the proximal end of the suture. The locking hub may also fluidly seal the catheter to a collection system and may include a luer connection. In some examples, the locking hub may require the use of an additional tool or “key” to actuate the locking mechanism. Further, the locking hub and/or the exit hole for the suture may be required to prevent fluid leaks and air ingress while also allowing the suture to be locked in place once the distal end of the catheter is curved or looped. Such locking hubs may be complex, unwieldy, and have a relatively high cost of goods sold (COGS). The present disclosure is directed towards alternative methods and systems for securing the proximal portion of a suture to maintain the distal end region of the catheter in a curved or looped configuration.

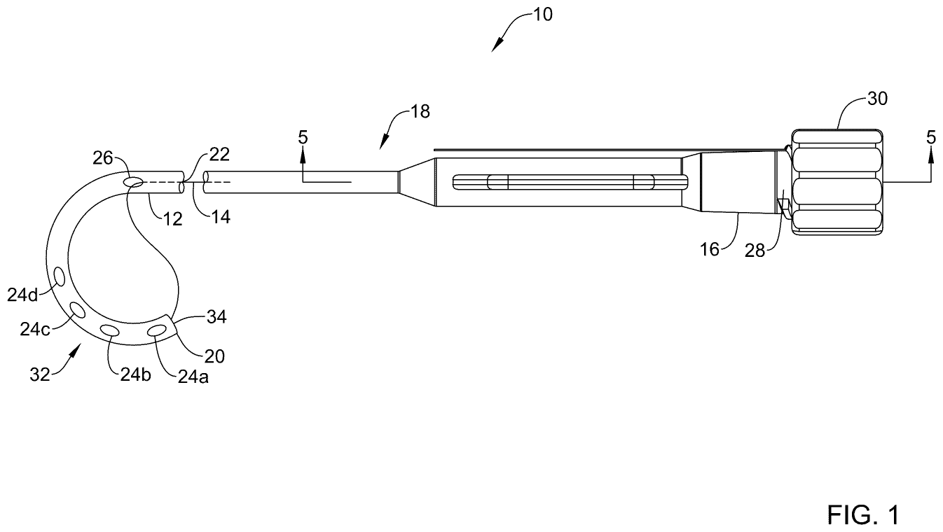

FIG. 1 is a side view of an illustrative locking catheter 10. In some examples, the locking catheter 10 may be a drainage catheter. The catheter 10 may generally include an elongate shaft 12, an elongate flexible member 14 (or flexible member 14), and a hub 16. The elongate shaft 12 may be a tubular member extending from a proximal end 18 coupled to the hub 16 and configured to remain outside the body to a distal end 20 configured to be inserted into the body. A lumen 22 may extend from the proximal end 18 to the distal end 20 of the elongate shaft 12. The elongate shaft 12 may have any cross-sectional shape, such as circular, rhombic, rectangular, oval, semicircular, or any other suitable shape. Further, a diameter or cross-sectional dimension of the elongate shaft 12 taken in a plane generally orthogonal to a longitudinal axis of the elongate shaft 12 may vary depending on one or more of the quantity of fluid to be extracted, fluid density, the size of the body cavity, the size of the cannula used to guide the elongate shaft 12 towards the desired location, etc. Further, the elongate shaft 12 may have a uniform cross-sectional shape and/or dimension from its proximal end 18 to the distal end 20. Alternatively, the cross-sectional dimension 20) of the elongate shaft 12 may increase, decrease, or otherwise vary from the proximal end 18 to the distal end 20. It is further contemplated that the cross-sectional shape of the elongate shaft 12 may vary along a length thereof.

The elongate shaft 12 may include one or more apertures 24a, 24b, 24c, 24d (collectively, 24) positioned along a distal end region 32 of the elongate shaft 12. The one or more apertures 24 may be longitudinally and/or circumferentially spaced from one another in a uniform or eccentric manner, as desired. The distal end 20 of the elongate shaft 12 may define a distal opening 34 colincar with a longitudinal axis of the elongate shaft 12. The one or more apertures 24 may extend through a thickness of a sidewall of the elongate shaft 12 to allow fluid to pass from the body into the lumen 22 of the elongate 30 shaft 12. The apertures 24 can be adapted to fit the needs of a given application. It is contemplated that the number of apertures 24 and/or their shape may vary based on the nature of the fluid. For example, if the fluid is viscous, the apertures 24 may be larger and/or more numerous. Alternatively, if the fluid has lower density or is less viscous, the apertures 24 may be smaller and/or fewer in number. It will be understood that the size and/or number of apertures 24 are not restrictive, and elongate shafts 12 with any size and/or number of apertures 24 are well within the scope of the present disclosure. For example, the elongate shaft 12 may include fewer than four or more than four apertures 24, as desired.

The elongate shaft 12 may be made of non-allergic or biocompatible material compatible with short or extended implantation in a patient's body. Such materials may include, for example, silicones and polyurethanes. It will be understood that any other suitable material may also be used. In one embodiment, the elongate shaft 12 may be coated with an anti-bacterial coating to inhibit bacterial growth on its surface. The anti-bacterial coating may contain an inorganic antibiotic agent, disposed in a polymeric matrix that adheres the antibiotic agent to the surface of the elongate shaft 12. Alternatively, or additionally, a drug release coating may be applied to the outer surface of the elongate shaft 12, which may assist in healing. Alternatively, or additionally, the elongate shaft 12 may include a lubricious coating to facilitate insertion and/or withdrawal of the elongate shaft 12.

A distal or first end (not explicitly shown) of the flexible member 14 may be coupled to a distal portion of the elongate shaft 12 and an intermediate region of the flexible member 14 may exit the elongate shaft 12 through the distal opening 34 or another aperture 24 to extend exterior to the elongate shaft 12. The flexible member 14 may then reenter the lumen 22 through a second aperture or opening 26 proximally spaced from the distal end 20 of the elongate shaft 12 and/or the one or more apertures 24. It is contemplated that the distance between the second aperture 26 and the distal end 20 of the elongate shaft 12 may be determined, at least in part, by a desired size of the pigtail or curve at the distal end region of the elongate shaft 12 when in a curved or looped configuration. Within the lumen 22, the flexible member 14 may extend proximally from the second aperture 26 to the proximal end 18 of the elongate shaft 12 where the flexible member 14 may exit the elongate shaft 12 at the proximal end 18 thereof.

In another embodiment, instead of attaching the distal or first end of the flexible member 14 adjacent to the distal end 20 of the elongate shaft 12, the flexible member 14 may be coupled to or adjacent to the junction of the elongate shaft 12 and the hub 16. From this proximally located fixation point, the flexible member 14 may extend distally through the lumen 22 towards the distal end 20 of the elongate shaft 12. The flexible member 14 may exit the lumen 22 through the distal opening 34 or another aperture 24 to extend exterior to the elongate shaft 12 and reenter the lumen 22 at the second aperture 26. Within the lumen 22, the flexible member 14 may then extend proximally from the second aperture 26 to the proximal end 18 of the elongate shaft 12 where the flexible member 14 may exit the elongate shaft 12 at the proximal end 18 thereof.

The flexible member 14 may be a suture thread made of nylon or other similar material of comparable strength. Alternatively, the flexible member 14 may be a thread or a flexible metal wire. To attach the distal or first end of the flexible member 14 to the distal end 20 of the elongate shaft 12 or at or adjacent to the junction of the elongate shaft 12 and the hub 16, suitable coupling techniques, such as tying, gluing, piercing, etc. may be used.

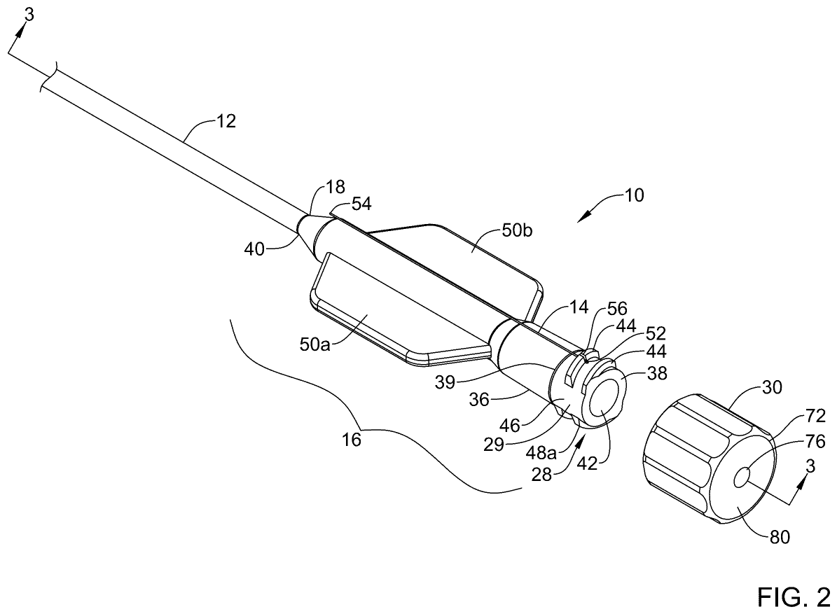

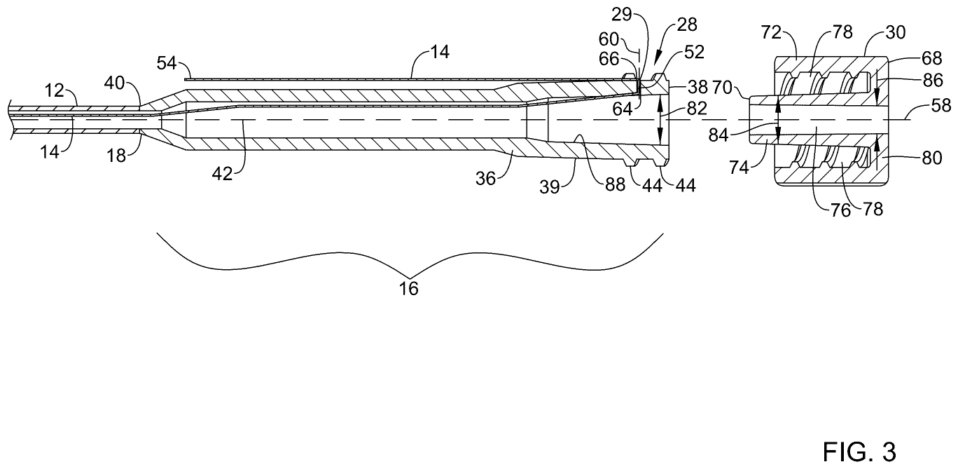

A first connection member 28 may be coupled to the hub 16 which in turn may be coupled to the proximal end 18 of the elongate shaft 12. In some embodiments, the first connection member 28 may be formed as a single monolithic structure with the hub 16. For example, the hub 16 and the first connection member 28 may be molded as a single piece. In other embodiments, the first connection member 28 may be formed as a separate 20) clement from the hub 16 and subsequently coupled thereto. For example, the first connection member 28 may be heat bonded, plastic welded, adhered, overmolded, etc. to the hub. A second connection member 30 may be releasably coupled to the first connection member 28. In some embodiments, the first connection member 28 and the second 25 connection member 30 may be a luer coupling mechanism. For example, the first and second connection members 28, 30 may be formed in compliance with ISO 80369-7. However, this is not required. The first and second connection members 28, 30 may take other structures, as desired. In some examples, the first connection member 28 may be a female luer lock and the second connection member 30 may be a male luer lock. The 30 reverse configuration is also contemplated in which the first connection member 28 is a male luer lock while the second connection member 30 is a female luer lock. The first connection member 28 and/or the second connection member 30 may be formed of any material, such as, but not limited to, nylons, polycarbonates, polyether block amides (e.g., such as, but not limited to, PEBAX®), urethanes, etc. The first connection member 28 may include an aperture 52 (see, for example, FIGS. 2 and 3) extending through a sidewall thereof to allow the flexible member 14 to pass from an interior of the hub 16 to an exterior of the hub 16, as will be described in more detail herein.

FIG. 2 is an exploded perspective view of a proximal end portion of the illustrative catheter 10 of FIG. 1. FIG. 3 is a cross-sectional view of the proximal end portion of the illustrative catheter 10 taken at line 3-3 of FIG. 2. In FIGS. 2 and 3, the second connection member 30 is uncoupled from the first connection member 28. The hub 16 may include a body portion 36 extending from a proximal end 38 to a distal end 40. The hub 16 may further include one or more wings 50a, 50b (collectively, 50) extending radially from the body portion 36. The one or more wings 50 may provide a gripping region for the user. The first connection member 28 may also include a body portion 29 extending from the proximal end 38 of the hub 16 to a distal end 39. In some examples, the body portion 29 of the first connection member 28 may form a portion of the body portion 36 of the hub 16, although this is not required. In other examples, the proximal end 38 of the hub 16 may correspond to the distal end 39 of the first connection member 28. A lumen 42 may extend through the first connection member 28 and through the hub 16 from the proximal end 38 to the distal end 40 of the hub 16. The lumen 42 of the hub 16 and/or first connection member 28 is in fluid communication with the lumen 22 of the elongate shaft 12. The distal end 40 of the hub 16 may be fixedly or releasably coupled to the proximal end 18 of the elongate shaft 12. In some examples, the hub 16 may be glued, adhered, overmolded, etc. to the elongate shaft 12. In other examples, the hub 16 may be mechanically coupled to the elongate shaft 12 for example, by a threaded connection, a snap fit, a friction fit, a keyed mechanism, etc. In yet other examples, the elongate shaft 12 and the hub 16 may be formed as a single monolithic structure.

An inner diameter 82 of the lumen 42 may vary from the proximal end 38 to the distal end 40 of the hub 16 and/or first connection member 28. It is contemplated that the inner diameter 82 may vary in a number of different manners. In one example, the inner diameter 82 may taper or reduce distally from a first diameter adjacent to the proximal end 38. The inner diameter 82 then may be substantially uniform over an intermediate region of the first connection member 28. The inner diameter 82 may then taper or reduce again to a smallest diameter adjacent to the distal end 40. In another example, the inner diameter 82 may include a substantially uniform taper from the proximal end 38 to the distal end 40. In other examples, the inner diameter 82 may be substantially constant from the proximal end 38 to the distal end 40. These are just some examples, other configurations of inner diameter changes may be used, as desired.

The first connection member 28 may further include one or more threads 44 extending radially from an outer surface of the body portion 36. In some embodiments, the one or more threads 44 may be adjacent to the proximal end 38 of the body portion 36. The one or more threads 44 may be interrupted or discontinuous. Said differently, a gap 46 may be positioned between helically adjacent thread regions 48a, 48b. However, this is not required. In some cases, the one or more threads 44 may be helically continuous about the body portion 36. While the first connection member 28 is illustrated as having two threads 44, in some examples, the first connection member 28 may have fewer than two threads (e.g., zero, less than one, one, etc.). It is further contemplated that the first connection member 28 may have more than two threads 44, as desired.

An aperture 52 may extend through a thickness of a sidewall of the body portion 29 of the first connection member 28 from a first interior end 64 to a second exterior end 66. In some examples, the first interior end 64 of the aperture 52 may include rounded surfaces or edges to reduce sharp edges. The flexible member 14 may exit the lumen 42 of the hub 16 and/or the first connection member 28 through the aperture 52 such that a proximal or second end 54 of the flexible member 14 is disposed exterior to the hub 16. This may allow the user to pull or otherwise actuate the proximal end 54 (or a region adjacent thereto) of the flexible member 14 that is located exterior to the lumen 22 of the elongate shaft 12 and the lumen 42 of the hub 16 and/or the first connection member 28 to deflect the distal end region 32 of the elongate shaft 12. In some embodiments, a longitudinal axis 60 of the aperture 52 may extend generally orthogonal to a longitudinal axis 58 of the elongate shaft 12. However, this is not required.

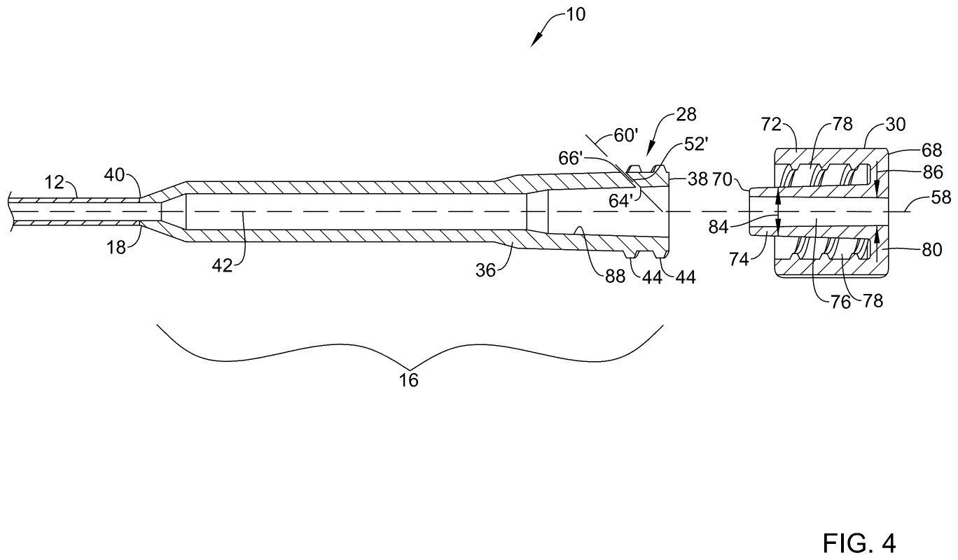

Referring to FIG. 4 which illustrates a cross-sectional view of a proximal portion of the illustrative catheter 10 having an alternative aperture 52′ extending through the first connection member 28. In some cases, a longitudinal axis 60′ of the aperture 52′ may extend at a non-orthogonal angle relative to the longitudinal axis 58 of the elongate shaft 12. In FIG. 4, the flexible member 14 has been omitted to more particularly illustrate the aperture 52′. However, the flexible member 14 may extend through the aperture 52′ in a similar manner to that show in FIGS. 2 and 3. In some cases, the longitudinal axis 60′ of the aperture 52′ may extend at an acute angle relative to the longitudinal axis 58 of the elongate shaft 12, as shown in FIG. 4. In this configuration, the second exterior end 66′ of the aperture 52′ of the first connection member 28 may be distal to the first interior end 64′ of the aperture 52′ of the first connection member 28. In another example, the longitudinal axis 60′ of the aperture 52′ may extend at an obtuse angle relative to the longitudinal axis 58 of the elongate shaft 12. In such a configuration, the second exterior end 66′ of the aperture 52′ of the first connection member 28 may be proximal to the first interior end 64′ of the aperture 52′ of the first connection member 28. In some examples, the first interior end 64′ of the aperture 52′ may include rounded surfaces or edges to reduce sharp edges.

Returning to FIGS. 2 and 3, in some cases, at least one of the threads 44 distal to the aperture 52 may include a notch or slot 56 configured to allow the flexible member 14 to pass distally beyond the one or more threads 44 such that the flexible member 14 does not interfere with the connection between the first and second connection members 28, 30. For example, the flexible member 14 may be disposed within the slot 56. In some embodiments, the slot 56 may be omitted.

The aperture 52 may be positioned in the range of about 0.09 inches (2.29 millimeters) to about 0.11 inches (2.79 millimeters) from the proximal end 38 of the first connection member 28 as measured from the proximal end 38 of the first connection member 28 to the center of the aperture 52. It is contemplated that if the aperture 52 extends at a non-orthogonal angle, the first interior end 64 of the aperture 52 of the first connection member 28 may be positioned in the range of about 0.09 inches (2.29 millimeters) to about 0.11 (2.79 millimeters) inches from the proximal end 38 of the first connection member 28 as measured from the proximal end 38 of the first connection member 28 to the center of the first interior end 62 of the aperture 52. However, in some cases, the aperture 52 may be less than 0.09 inches (2.29 millimeters) or more than 0.11 (2.79 millimeters) inches from the proximal end 38 of the first connection member 28. A diameter of the aperture 52 may be sized to allow the flexible member 14 to pass therethrough. For example, if the flexible member 14 has a diameter of about 0.008 inches (0.203 millimeters), the diameter of the aperture 52 may be about 0.010 inches (0.254 millimeters) to about 0.014 inches (0.356 millimeters). It is contemplated that the diameter of the aperture 52 may vary based on the diameter or size of the flexible member 14 and the diameter of the aperture 52 may be less than 0.010 inches (0.254 millimeters) or greater than 0.014 inches (0.356 millimeters). In some embodiments, the aperture 52 may be formed during molding of the first connection member 28. In other embodiments, the aperture 52 may be formed after formation of the first connection member 28, using techniques such as, but not limited to, drilling, piercing, puncturing, etc.

The second connection member 30 may extend from a proximal end 68 to a distal end 70. The second connection member 30 may include an outer coupling member 72 and an inner tubular member 74. The inner tubular member 74 may be coaxially disposed within the outer coupling member 72. Further, the inner tubular member 74 may be coupled to the outer coupling member 72 adjacent the proximal end 68 of the second connection member 30 via a proximal annular wall 80. A lumen 76 may extend through the inner tubular member 74 from the proximal end 68 to the distal end 70 of the second connection member 30. The outer diameter 84 of the inner tubular member 74 may decrease from a proximal end region to the distal end 70 thereof. In some cases, the outer diameter 84 may be sloped or tapered to form a friction fit with the inner surface 88 of the first connection member 28 to form a fluid tight (e.g., air-light and liquid-tight) seal therewith. The outer diameter 84 of the inner tubular member 74 may vary in other manners or be substantially uniform as desired. In some embodiments, an inner diameter 86 of the inner tubular member 74 may increase from the proximal end region to the distal end 70. The inner diameter 86 of the inner tubular member 74 may vary in other manners or be substantially uniform as desired. In some cases, a wall thickness of the inner tubular member 74 may vary over a length of the inner tubular member 74, although this is not required.

The outer coupling member 72 may include one or more internal helical recesses 78 configured to threadably engage the one or more threads 44 of the first connection member 28. The outer coupling member 72 may be configured to be disposed over the outer surface of the proximal end region of the first connection member 28 while the inner tubular member 74 may be configured to be received within the lumen 42 of the first connection member 28 when the second connection member 30 is coupled with the first connection member 28. While the first and second connection members 28, 30 are shown and described as having a threaded engagement which can allow the second connection member 30 to be selectively and removably attached to the first connection member 28, other connection mechanisms may be used as desired. Some illustrative connection mechanisms may include, but are not limited to, friction fits, snap fits, a bayonet style connection, etc.

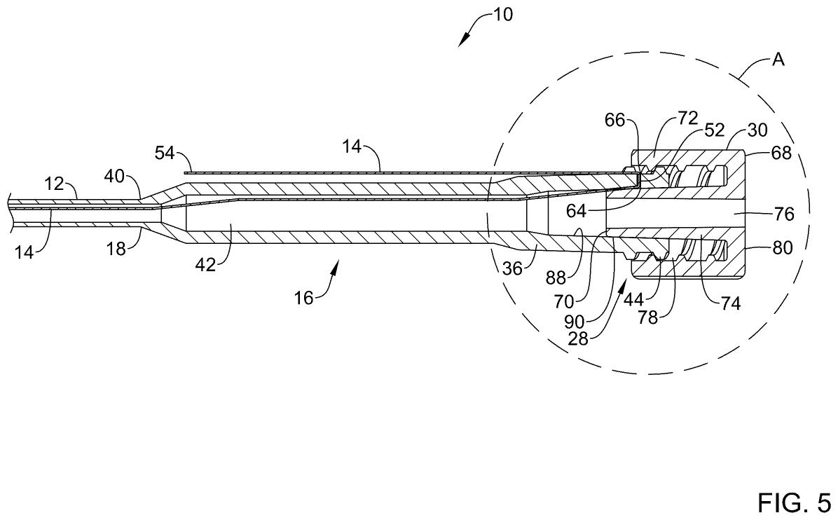

FIG. 5 is a cross-sectional view of the proximal portion of the catheter 10, taken at line 5-5 of FIG. 1. In FIG. 5, the second connection member 30 is assembled with or connected to the first connection member 28. As can be seen in FIG. 5, the internal grooves 78 of the second connection member 30 threadably engage the external threads 44 of the first connection member 28 to releasably secure the second connection member 30 to the first connection member 28. Further, when the second connection member 30 is coupled with the first connection member 28, the inner tubular member 74 is disposed within the lumen 42 of the first connection member 28 and the lumen 76 of the second connection member 30 is in fluid communication with the lumen 42 of the hub 16 and/or the first connection member 28. An outer surface 90 of the inner tubular member 74 may engage the inner surface 88 of the first connection member 28 to form a fluid-tight seal therewith. The distal end 70 of the inner tubular member 74 may extend distally beyond the aperture 52 in the first connection member 28. As the inner tubular member 74 is inserted into the lumen 42 of the first connection member 28, the flexible member 14 may be pinched or squeezed between the outer surface 90 of the inner tubular member 74 adjacent the distal end 70 thereof and the inner surface 88 of the first connection member 28 thus preventing the actuation or movement of the flexible member 14. In some examples, the first connection member 28 and/or the second connection member 30 may be formed from a softer plastic to accommodate the flexible member 14 and/or to improve sealing. It is contemplated that the inner tubular member 74 may form a fluid-tight seal with the interior end 64 of the aperture 52 of the first connection member 28 to prevent air, gases, liquids, fluids, etc. from entering or egressing the lumen 42 via the aperture 52. However, this is not required. For example, a fluid-tight seal may be formed between the outer surface 90 of the inner tubular member 74 and the inner surface 88 of the first connection member 28 adjacent to the distal end 70 of the inner tubular member 74. This may prevent air, gas, fluid, etc. from the lumens 22, 42 from reaching the aperture 52 and/or air, gas, fluids, etc., from the atmosphere from entering the lumens 22, 42 via the aperture 52. Further, a fluid-tight seal may be formed between the outer surface 90 of the inner tubular member 74 and the inner surface 88 of the first connection member 28 over a length proximal to the aperture 52 to form a fluid-tight seal between the first connection member 28 and the second connection member 30.

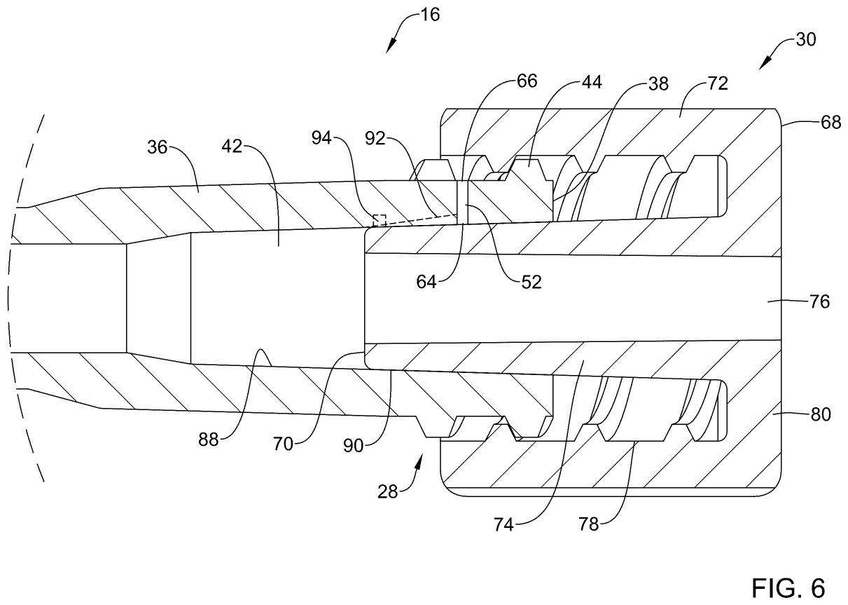

FIG. 6 is an enlarged cross-sectional view of the connection between the first connection member 28 and the second connection member 30 with the flexible member 14 removed for clarity, identified as region A in FIG. 5. In some embodiments, the first connection member 28 may include a slot or channel 92 formed in the inner surface 88 thereof. However, this is not required. The channel 92 may extend distally from the first interior end 64 of the aperture 52 and may be configured to receive the flexible member 14 such that the inner tubular member 74 can be distally advanced within the lumen 42 of the first connection member 28 with little interference from the flexible member 14. If so provided, a distal end of the channel 92 may be proximal to the distal end 70 of the inner tubular member 74 when the second connection member 30 is coupled with the first connection member 28 in a user configuration. It is contemplated that such a channel 92 may be manufactured by the use of a collapsible core-pin. In some cases, the first connection member 28 may include an optional internal notch 94 configured to be positioned adjacent to the distal end 70 of the inner tubular member 74 when the second connection member 30 is coupled to the first connection member 28 in a use configuration. The notch 94 may cause the compression force exerted on the flexible member 14 to be lessened. It is contemplated that this may allow more flexibility in the location of the aperture 52.

When the second connection member 30 is coupled with the first connection member 28, the flexible member 14 and thus the distal end region 32 of the elongate shaft 12 may be locked into a position (e.g., proximal and/or distal movement of the flexible member 14 is precluded). To curl the distal end region 32 of the elongate shaft 12, the user may uncouple the second connection member 30 from the first connection member 28 to free or unlock the flexible member 14. In some cases, the second connection member 30 need not be entirely removed from engagement with the first connection member 28 to allow for movement of the flexible member 14. For example, the second connection member 30 may be proximally displaced without completely uncoupling the second connection member 30 from the first connection member 28. In other examples, the second connection member 30 may be free from contact with the first connection member 28 as the flexible member 14 is actuated.

Once the flexible member 14 is unsecured, the user may pull or otherwise actuate the flexible member 14 to bend the distal end region 32 of the elongate shaft 12 into a pigtail loop or a curved configuration. Once the desired shape of the distal end region 32 of the elongate shaft 12 has been obtained, the second connection member 30 may be coupled to or tightened to the first connection member 28 to secure the flexible member 14 between the outer surface 90 of the inner tubular member 74 and the inner surface 88 of the first connection member 28. It is contemplated that the user may maintain the flexible member 14 in the desired position as the second connection member 30 is coupled to or tightened to the first connection member 28 to maintain the configuration of the distal end region 32 of the elongate shaft 12.

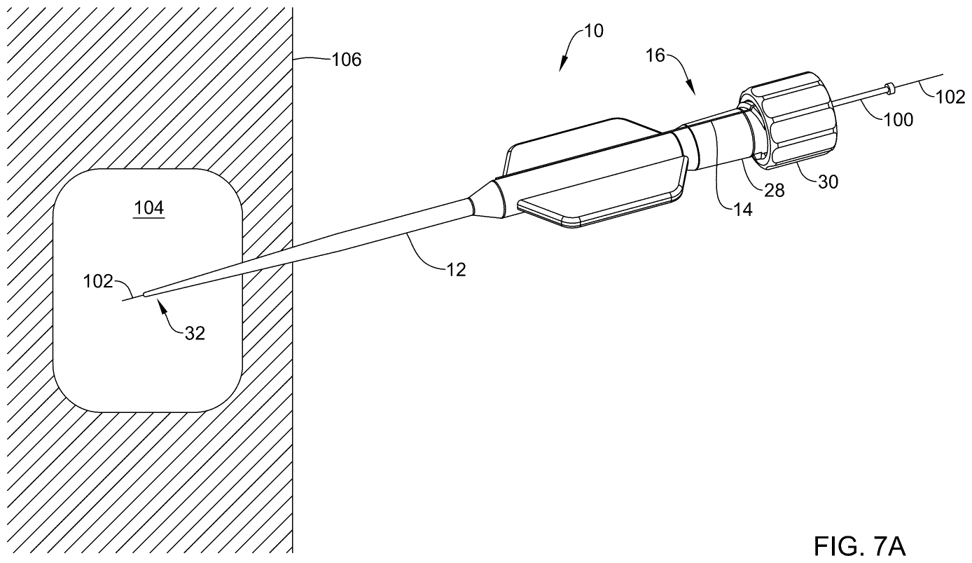

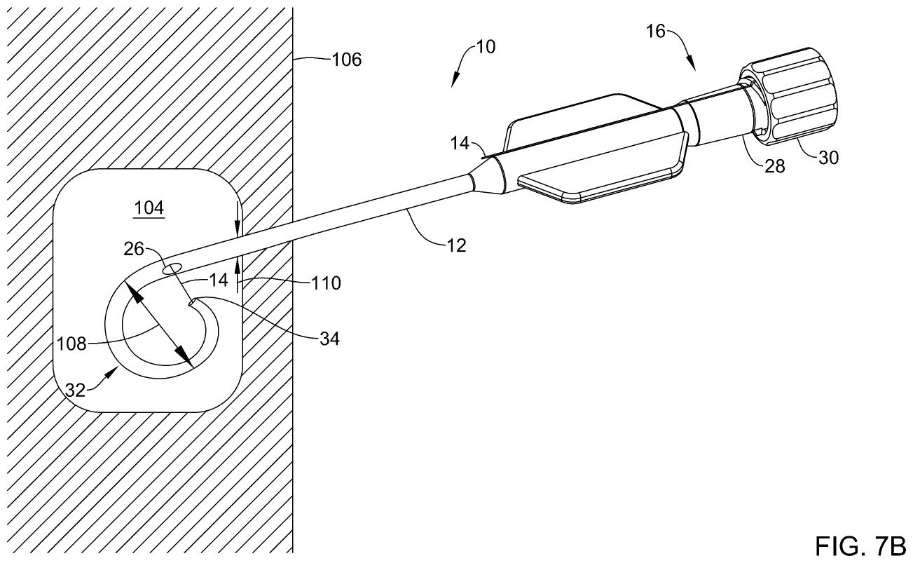

FIG. 7A illustrates a schematic view of the catheter 10 disposed within a patient in an unlocked or insertion configuration. The distal end region 32 of the elongate shaft 12 may be straightened with a stiffening stylet 100 may be disposed within the lumen 22 of the elongate shaft 12. The elongate shaft 12 and/or stiffening stylet 100 may be advanced over a guidewire 102 and inserted into a fluid collection within the viscera 104. The stiffening stylet 100 and the guidewire 102 may be removed from the patient's body 106 while leaving the distal end region 32 of the elongate shaft 12 disposed within the viscera 104. During insertion of the catheter 10, the second connection member 30 may be secured to the first connection member 28, although this is not required.

FIG. 7B illustrates a schematic view of the catheter 10 disposed within a patient in a locked or deployed configuration. To move the distal end region 32 of the elongate shaft 12 into a curved and locked configuration (FIG. 7B) from a straightened delivery configuration (FIG. 7A), the second connection member 30 may be uncoupled from the first connection member 28. The proximal end region of the flexible member 14 may then be pulled to draw the distal end 20 of the elongate shaft 12 towards the second aperture 26 to form a loop or a curve in the distal end region 32 of the elongate shaft 12. It is contemplated that the loop or curve in the distal end region 32 of the elongate shaft 12 may have an outer dimension 108 that is greater than the dimension 110 of the opening through the body 106 to prevent accidental removal of the elongate shaft 12 from the body 106. Once the flexible member 14 has been actuated and the distal end region 32 of the elongate shaft 12 is in the desired configuration, the second connection member 30 may be coupled to the first connection member 28 while the user holds the flexible member 14 in the desired position. When the outer surface 90 distal end region of inner tubular member 74 of the second connection member 30 engages the inner surface 88 of the first connection member 28, the flexible member 14 may be pinched or squeezed between the surfaces 88, 90 of the first and second connection members 28, 30 preventing movement of the flexible member 14 and locking the distal end region 32 of the elongate shaft 12 into the curved or looped configuration. In some embodiments, the excess length of the flexible member 14 which extends from the hub 16 may be trimmed. However, this is not required.

The hub 16 described herein may have numerous advantageous to existing locking systems. For example, the mechanism to lock the flexible member 14 and the fluid coupling mechanism (e.g., the hub 16) are combined into a single system. It is contemplated that the flexible member 14 and hence the distal end region 32 of the elongate shaft 12 will remain locked or secured until the second connection member 30 is uncoupled and/or proximally retracted from the first connection member 28. Alternatively, the distal end region 32 can assume a straightened configuration if the elongate shaft 12 is cut (thus cutting the flexible member 14). Further, the flexible member 14 and the distal end region 32 of the elongate shaft 12 may be unlocked (and the distal end region 32 allowed to return to a straightened configuration) by uncoupling and/or proximally retracting the second connection member 30 from the first connection member 28. The flexible member 14 may be free from travel restrictions which allow the elongate shaft 12 to be moved between the curved and straightened configuration. As a clinician is familiar with the operation of a luer coupling mechanism, the locking and unlocking of the first and second connection members 28, 30 may be intuitive, does not require additional tools or devices to operate, and may be performed without looking at the first and second connection members 28, 30 as they are coupled and/or uncoupled. Further, there may be no interference between the cannula or stiffening stylet 100 and the flexible member 14 or hub 16. As described above, the hub 16 secures the flexible member 14 while also preventing the ingress of air or the leakage of fluid. Contrast, saline, or other fluids may be injected into or aspirated from the catheter 10 with the distal end region 32 of the elongate shaft 12 in an unlocked configuration (e.g., straightened) or locked configuration (e.g., curved or looped). Further, allowing the flexible member 14 to exit the hub 16 at a location other than the proximal end of the hub 16 may reduce leaks. The present hub 16 may be ergonomic and non-traumatic to the patient. For example, the present hub 16 may reduce extra components and thus the weight of the catheter 10 as well has having a smooth surface which may reduce trauma to the patient and/or the patient's skin. In addition to the reducing the overall weight of the device, the reduction of components may also reduce the COGS of the catheter 10. Finally, the hub 16 may be compatible with alcohol as no components have been added.

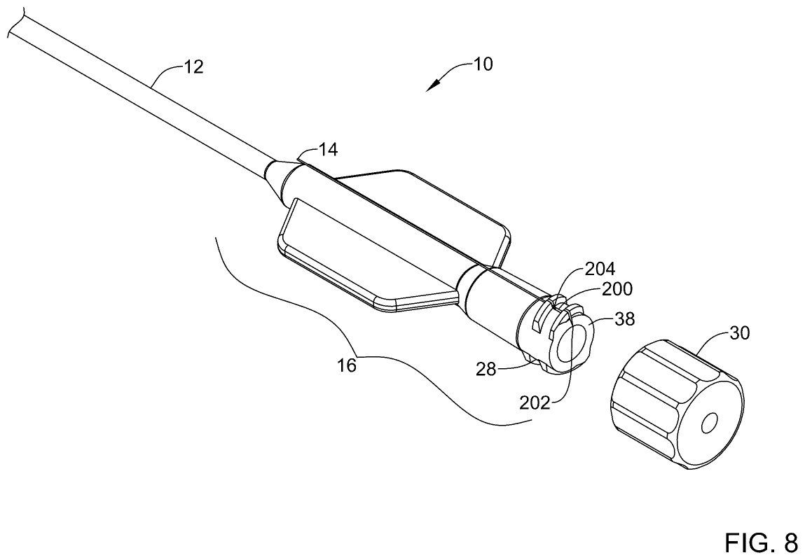

FIG. 8 is a perspective view of the proximal end portion of the catheter 10 with the second connection member 30 uncoupled from the first connection member 28 with the catheter 10 having an alternative exit means for the flexible member 14. In place of the aperture 52, the first connection member 28 may include a slot 200. The slot 200 may extend from a proximal end 202 adjacent to the proximal end 38 of the first connection member 28 to a distal end 204. The slot 200 may have a length such that the distal end 204 thereof is positioned in the range of about 0.09 inches (2.29 millimeters) to about 0.11 (2.79 millimeters) inches from the proximal end 38 of the first connection member 28. However, this is not required. The distal end 204 of the slot 200 may be less than 0.09 inches (2.29 millimeters) or more than 0.11 (2.79 millimeters) inches from the proximal end 38 of the first connection member 28. The slot 200 may have width sized to allow the flexible member 14 to pass therethrough. For example, if the flexible member 14 has a diameter of about 0.008 inches (0.203 millimeters), the width of the slot 200 may be about 0.010 inches (0.254 millimeters) to about 0.014 inches (0.356 millimeters). It is contemplated that the width of the slot 200 may vary based on the diameter or size of the flexible member 14 and the width of the slot 200 may be less than 0.010 inches (0.254 millimeters) or greater than 0.014 inches (0.356 millimeters). In some embodiments, the slot 200 may be formed during molding of the first connection member 28. In other embodiments, the slot 200 may be formed after formation of the first connection member 28, using techniques such as, but not limited to, drilling, piercing, puncturing, etc.

The slot 200 may extend through a thickness of the sidewall of the first connection member 28 (e.g., from the interior surface 88 to an exterior surface). The flexible member 14 may be passed through the slot 200 such that the proximal end 54 or proximal end region of the flexible member 14 is exterior to the lumen 42 of the hub 16 and/or the first connection member 28. The proximal end 54 of the flexible member 14 may be moved distal to the distal end 204 of the slot 200 to bring the portion of the flexible member 14 in contact with a distal surface of the slot 200. This may prevent the flexible member 14 from exiting the proximal end 68 of the second connection member 30 which may help limit leakage.

The second connection member 30 may engage the first connection member 28 in a similar manner to that described above with respect to FIGS. 1-7B. For example, the exterior surface 90 of the inner tubular member 74 may be configured to engage the interior surface 88 of the first connection member 28 to pinch the flexible member 14 therebetween and lock it in place (e.g., proximal and/or distal movement of the flexible member 14 is precluded). Further, the exterior surface 90 of the inner tubular member 74 may be configured to engage the interior surface 88 of the first connection member 28 to fluidly seal the slot 200. For example, the inner tubular member 74 may form a fluid-tight seal with the interior of the slot 200 of the first connection member 28 to prevent air, gases, liquids, fluids, etc. from entering or egressing the lumen 42 via the slot. However, this is not required. For example, a fluid-tight seal may be formed between the outer surface 90 of the inner tubular member 74 and the inner surface 88 of the first connection member 28 adjacent to the distal end 204 of the slot 200 and/or the distal end 70 of the inner tubular member 74. This may prevent air, gas, fluid, etc. from the lumens 22, 42 from reaching the aperture 52 and/or air, gas, fluids, etc., from the atmosphere from entering the lumens 22, 42 via the slot 200. Similarly, the fluid-tight seal between the first connection member 28 and the second connection member 30 may prevent fluid from leaking from the lumen 42 of the first connection member 28 via the slot 200.

The materials that can be used for the various components of the catheter assembly, and/or other devices disclosed herein may include those commonly associated with medical devices. For simplicity purposes, the following discussion makes reference to the catheter assembly and its related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfonc, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the catheter assembly and its related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter assembly and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course. defined in the language in which the appended claims are expressed.

What is claimed is:

1. A locking catheter, comprising:
an elongate shaft extending from a proximal end to a distal end and defining a lumen extending from the proximal end to the distal end;
a hub defining a lumen extending from a proximal end to a distal end thereof and defining a lumen including extending from the proximal end to the distal end, the hub including a first connection member having a body portion extending from a proximal end to a distal end and defining an aperture extending through a sidewall of the body portion of the first connection member from an inner surface to an outer surface thereof, the hub coupled to the proximal end of the elongate shaft;
a second connection member extending from a proximal end to a distal end and having an outer coupling member and an inner tubular member, the inner tubular member defining a lumen extending from the proximal end to the distal end of the second connection member, the second connection member configured to be releasably coupled to the first connection member; and
a flexible member extending from a proximal end to a distal end, the distal end of the flexible member coupled to the elongate shaft, the flexible member extending through at least a portion of the lumen of the elongate shaft and a portion of the lumen of the hub, the proximal end of the flexible member extending through the aperture of the first connection member such that the proximal end of the flexible member is exterior to the first connection member;
wherein when the second connection member is coupled with the first connection member, the flexible member is secured between an inner surface of the first connection member and an outer surface of the inner tubular member of the second connection member.

2. The locking catheter of claim 1, wherein when the second connection member is coupled with the first connection member, the second connection member and the first connection member form a fluid-tight seal.

3. The locking catheter of claim 1, wherein the first connection member comprises a female luer lock.

4. The locking catheter of claim 1, wherein the second connection member comprises a male luer lock.

5. The locking catheter of claim 1, wherein the aperture of the first connection member is in the range of about 0.09 inches (2.29 millimeters) to about 0.11 inches (2.79 millimeters) from the proximal end of the first connection member.

6. The locking catheter of claim 1, wherein the aperture of the first connection member has a diameter in the range of about 0.010 inches (0.254 millimeters) to about 0.014 inches (0.356 millimeters).

7. The locking catheter of claim 1, further comprising a channel formed in the inner surface of the first connection member.

8. The locking catheter of claim 7, wherein the channel extends distally from the aperture of the first connection member.

9. The locking catheter of claim 1, wherein a longitudinal axis of the aperture of the first connection member extends generally orthogonal to a longitudinal axis of the elongate shaft.

10. The locking catheter of claim 1, wherein a longitudinal axis of the aperture of the first connection member extends at a non-orthogonal to a longitudinal axis of the elongate shaft.

11. The locking catheter of claim 1, wherein actuation of the flexible member is configured to move a distal end region of the elongate shaft into a curved configuration.

12. The locking catheter of claim 1, wherein the distal end of the flexible member is coupled to the elongate shaft adjacent to the distal end of the elongate shaft.

13. The locking catheter of claim 1, wherein the distal end of the flexible member is coupled to the elongate shaft adjacent to the proximal end of the elongate shaft.

14. The locking catheter of claim 1, further comprising a plurality of apertures extending through a sidewall of the elongate shaft, the plurality of apertures adjacent to the distal end of the elongate shaft.

15. The locking catheter of claim 1, wherein the outer coupling member of the second connection member is configured to threadably engage the first connection member.

16. A locking catheter, comprising:
an elongate shaft extending from a proximal end to a distal end and defining a lumen extending from the proximal end to the distal end;
a hub defining a lumen extending from a proximal end to a distal end thereof and defining a lumen including extending from the proximal end to the distal end, the hub including a female luer lock having a body portion extending from a proximal end to a distal end and defining an aperture extending through a sidewall of the body portion of the female luer lock from an inner surface to an outer surface thereof, the hub coupled to the proximal end of the elongate shaft;
a male luer lock extending from a proximal end to a distal end and having an outer coupling member and an inner tubular member, the inner tubular member defining a lumen extending from the proximal end to the distal end of the male luer lock, the male luer lock configured to be releasably coupled to the female luer lock; and
a flexible member extending from a proximal end to a distal end, the distal end of the flexible member coupled to the elongate shaft, the flexible member extending through at least a portion of the lumen of the elongate shaft and a portion of the lumen of the hub, the proximal end of the flexible member extending through the aperture of the female luer lock such that the proximal end of the flexible member is exterior to the female luer lock;
wherein when the male luer lock is coupled with the female luer lock, the flexible member is secured between an inner surface of the female luer lock and an outer surface of the inner tubular member of the male luer lock.

17. The locking catheter of claim 16, wherein the aperture of the female luer lock is distal to the proximal end of the female luer lock.

18. The locking catheter of claim 16, wherein when the male luer lock is coupled with the female luer lock, the male luer lock and the female luer lock form a fluid-tight seal.

19. The locking catheter of claim 16, wherein the aperture of the female luer lock is in the range of about 0.09 inches (2.29 millimeters) to about 0.11 inches (2.79 millimeters) from the proximal end of the female luer lock.

20. A locking catheter, comprising:
an elongate shaft extending from a proximal end to a distal end and defining a lumen extending from the proximal end to the distal end;
a hub defining a lumen extending from a proximal end to a distal end thereof and defining a lumen including extending from the proximal end to the distal end, the hub including a first connection member coupled to the proximal end of the elongate shaft, the first connection member having a body portion extending from a proximal end to a distal end and defining a slot extending through a thickness of a sidewall of the body portion, the slot extending distally from the proximal end of the first connection member;
a second connection member extending from a proximal end to a distal end and having an outer coupling member and an inner tubular member, the inner tubular member defining a lumen extending from the proximal end to the distal end of the second connection member, the second connection member configured to be releasably coupled to the first connection member; and
a flexible member extending from a proximal end to a distal end, the distal end of the flexible member coupled to the elongate shaft, the flexible member extending through at least a portion of the lumen of the elongate shaft and a portion of the lumen of the hub, the proximal end of the flexible member extending through the slot of the first connection member such that the proximal end of the flexible member is exterior to the first connection member;
wherein when the second connection member is coupled with the first connection member, the flexible member is secured between an inner surface of the first connection member and an outer surface of the inner tubular member of the second connection member.
